(19) United States
(12) Patent Application Publication
(10) Pub. No.: US 2007/0243163 A1
(43) Pub. Date: Oct. 18, 2007

(54) Respiratory tract delivery of interferon-tau

(76) Inventor: Chih-Ping Liu, San Francisco, CA (US)

Correspondence Address: PERKINS COIE LLP, P.O. BOX 2168, MENLO PARK, CA, 94026, US

(21) Appl. No.: 11/707,333

(22) Filed: Feb. 16, 2007

Related U.S. Application Data
(60) Provisional application No. 60/774,328, filed on Feb. 17, 2006.

(51) Int. Cl. A61K 38/21; A61P 17/00; A61P 37/00; A61P 43/00

(52) U.S. Cl. 424/85.6; 424/85.4; 424/85.7

(57) ABSTRACT

Methods for treating systemic or local diseases or conditions by administering an interferon as therapeutic agent to one or several regions of the respiratory tract are provided. In one embodiment, the interferon is interferon-tau.

### CROSS-REFERENCE TO RELATED APPLICATIONS

This application claims the benefit of U.S. Provisional Application No. 60/774,328, filed Feb. 17, 2006, incorporated herein by reference in its entirety.

### TECHNICAL FIELD

The present invention relates to respiratory delivery of interferon-tau (interferon-τ) for treatment of systemic or local diseases, conditions, illness or infections. In particular, the delivery methods target nasal passages, the nasal cavity, pulmonary tissue and other regions within the respiratory tract for administration of therapeutically effective doses of interferon-τ.

### BACKGROUND

Interferon-τ was discovered originally as a pregnancy recognition hormone produced by the trophectoderm of ruminant conceptuses (Imakawa, K. et al, Nature, 330:377-379, (1987); Bazer, F. W. and Johnson, H. M., Am. J. Repro. Immunol., 26:19-22, (1991)). The interferon-τ gene is restricted to ruminants, including cattle, sheep, and goats, (Alexenko, A. P. et al., J. Interferon and Cytokine Res., 19:1335-1341, (1999)) but interferon-τ has biological activity in cells belonging to other species including humans and mice (Pontzer, C. H. et al., Cancer Res., 51:5304-5307, (1991); Alexenko, A. P. et al., J. Interferon and Cytokine Res., 20:817-822, (2000)). For example, interferon-τ has been demonstrated to possess antiviral, (Pontzer, C. H. et al., Biochem. Biophys. Res. Commun., 152:801-807(1988)), antiproliferative, (Pontzer, C. H., et al., 1991) and immunoregulatory activities (Assal-meliani, A., Am. J. Repro. Immunol., 33: 267-275 (1995)).

Although interferon-τ is considered a Type I interferon, being acid-stable and displaying many of the activities associated with classic Type I interferons, such as interferon-α and interferon-β, there are significant differences between interferon-τ and the other Type I interferons. The most prominent difference is that interferon-τ plays a role in establishing maternal recognition and a physiological response to pregnancy in ruminant species. The other interferons have no similar activity in pregnancy recognition. Also different is viral induction. All Type I interferons except interferon-τ are induced readily by virus and dsRNA (Roberts, et al., Endocrine Reviews, 13:432 (1992)). When induced, the expression of interferon-α and interferon-β is transient, lasting approximately a few hours. In contrast, interferon-τ synthesis, once induced, is maintained over a period of days (Godkin, et al., J. Reprod. Fert., 65:141 (1982)). On a per-cell basis, 300-fold more interferon-τ is produced than other Type I interferons (Cross, J. C. and Roberts, R. M., Proc. Natl. Acad. Sci. USA 88:381-3821 (1991)).

In addition to the above functional differences, interferon-τ is significantly less cytotoxic when administered as a drug. Interferons have found widespread application in the treatment of various diseases and conditions, such as cancer, viral infection, MS and others. However, the usefulness of the first INF proteins used, interferon-α and interferon-β, has been limited by toxicity and side effects due to these drugs.

Determining that interferon-τ (INF-τ) and hybrid INF/INF-τ proteins achieved therapeutic benefit without the cytotoxicity of the native human proteins was a significant development in this field. U.S. Pat. No. 5,906,816 (Soos et al.) taught IFN-τ was less toxic in vivo than either IFN-α or IFN-β and useful for therapeutic treatments of, e.g., autoimmune diseases.

Other patents have also disclosed that modified forms of the protein, analogs and fusion products of IFN-τ, display reduced cytotoxicity. U.S. Pat. Nos. 5,939,286 and 6,174,996 (both to Johnson et al.) disclosed fusion polypeptides comprising an N-terminal region having an IFN-τ sequence and a C-terminal region having a Type I interferon sequence such as IFN-α or IFN-β. The fusion protein also exhibited lower cytotoxicity than native human Type I interferons. U.S. Patent Application Pub. No. 2003/0130486 (Villarete et al.) disclosed that appending the C-terminal region of IFN-τ to a non-τ interferon (including replacing a portion of the C-terminal region of the non-τ interferon) produced a therapeutic with lower toxicity. U.S. Pat. No. 6,204,022 (Johnson et al.) describes a method for producing a low toxicity analog of INF-α comprising substituting one or more amino acids within the first 27 residues of the N-terminal with amino acids representative of an INF-τ sequence at that position.

A limiting factor in the use of interferon-τ, as well as analogs and fusion products in general, is the difficulty in providing an effective means of delivery to the body as well as the achievement of the desired biodistribution. Various routes of delivery and methods of treatment have been disclosed for interferon, as for example in U.S. Pat. No. 5,906,816 to Soos et al. This patent disclosed administration of interferon-τ by intravenous or intramuscular injection to achieve systemic delivery. However, when given parenterally, the therapeutic protein interacts with plasma proteins and blood cells, affecting the availability of the drug.

Interferon-τ has also been administered orally for treatment of systemic autoimmune conditions and viral infections (Soos, J. M. et al., J. Neuroimmunology, 75:43-50 (1997); Soos, J. M. et al., J. Immunology, 169(5):2231-2235 (2002); Nakajima, A. et al., J. Interferon Cytokine Res., 22:397-401 (2002)), and was disclosed in U.S. Pat. Nos. 6,942,854 and 6,372,206, both to Soos et al. However it is suspected that this route may be problematic due to proteolysis in the stomach, where the acid conditions can destroy the molecule before reaching its intended target. An improved method for oral delivery has been disclosed in U.S. Publication No. 2003/0219405, whereby drug administration is coordinated with the fasting state of the patient.

Despite these potential improvements much effort continues to be focused on methods of delivering proteins to the body. One general route of interest is the absorption of therapeutic agents through the soft mucosal tissues in the interior regions of the body. In principle, the agents can pass directly into systemic circulation while avoiding a first pass through the digestive tract and liver. Because protein- and peptide-based medications are virtually completely degraded by the gut and liver, transmembrane delivery into the bloodstream holds the promise of an effective and efficient administration methods.

The efficacy of transmucosal delivery depends on many factors, including the amount of mucosal surface exposed to medication and the time over which the medication remains present and available on the mucosal surface. The respiratory tract presents a large surface area region adapted to the movement of particles across the membrane and into the circulatory system and has been used for the delivery of drugs into the body. In addition to being a target for local administration of a therapeutic, the upper and lower respiratory tracts are an alternative, non-invasive route to systemic administration. There is not yet, however, a suggestion in the art to administer interferon-τ and its variants and fusion products via the respiratory tract for the treatment of interferon-responsive diseases and conditions.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a treatment method comprising administering an interferon, and in particular, interferon-τ or a variant interferon protein, to the respiratory tract of a patient is provided. In one embodiment, the patient is suffering from a condition, disease, illness or infection which is systemic or localized, that is responsive to a Type I interferon. In other embodiments, the interferon is a Type I interferon, such as interferon-alpha, interferon-tau, or interferon-omega. In another embodiment, the interferon in is a Type II interferon, such as interferon-beta.

In another aspect, the interferon, and in particular interferon-τ or a variant interferon protein, is administered via the nose in the form of a solution, spray, drops, suspension, or aerosol. These preparations can be formulated, in other embodiments, for sustained release. Swellable polymers and other base compositions are also contemplated, such that when the compositions is placed in contact with the lining in the nasal cavity, the therapeutic agent loaded in the composition is released into the adjacent tissue. In one embodiment, the composition administered further comprises an absorption enhancement agent and/or an enzyme inhibitor.

In yet another aspect, the interferon, and in particular interferon-τ or variant interferon protein, is administered via the mouth and into the respiratory tract in the form of an aerosol or dry powder. In one embodiment, the composition administered further comprises an absorption enhancement agent and/or an enzyme inhibitor.

In one embodiment, the therapeutic dose is administered using a metered dose inhaler, a dry powder inhaler, a nebulizer or a dropper.

In one embodiment, the interferon-τ has a certain degree of identity to the protein identified herein as SEQ ID NO:2.

In another embodiment, the variant interferon has a certain degree of identity to the protein identified herein as SEQ ID NO:3.

In another embodiment, the interferon-τ is a hybrid or fusion protein. In a particularly preferred embodiment, the N-terminal region sequence is derived from interferon-τ.

In another embodiment, the interferon-τ is formulated with histidine, for increased stability and/or solubility in aqueous formulations.

In another embodiment, the interferon-based therapeutic agent is conjugated with a hydrophilic polymer such that the clearance time from the body is longer than non-conjugated therapeutic agents.

In some embodiments, the condition responsive to interferon therapy is a disease selected from multiple sclerosis, type I (insulin dependent) diabetes mellitus, lupus erythematosus, amyotrophic lateral sclerosis, Crohn's disease, rheumatoid arthritis, stomatitis, asthma, uveitis, allergies, and psoriasis. In a particular embodiment, the interferon is interferon-tau. In some embodiments, the administration of interferon-tau causes systemic suppression of T-cell immunity. In particular embodiments, the administration of interferon-tau causes an increase in the levels of a gene selected from TGFβ, FOXP3, and IFNAR1.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1C depict graphs showing the results of gene expression analysis in spleen tissues obtained from mice treated with different amounts of IFN-τ (i.e., 3.125, 12.5, or 50 KU) or mock-treated with PBS. The data are shown in Table 2. Expression levels are normalized for those of hprt. The relative levels of (A) TGFβ, (B) FOXP3, and (C) IFNAR1 are shown.

### BRIEF DESCRIPTION OF THE SEQUENCES

SEQ ID NO:1 corresponds to an amino acid sequence of mature ovine interferon-τ (IFN-τ; oTP-1; GenBank Accession No. CM68396; PID g1358).

SEQ ID NO:2 corresponds to an amino acid sequence of mature ovine IFN-τ, where the amino acid residues at positions 5 and 6 of the sequence are modified relative to the sequence of SEQ ID NO:1.

SEQ ID NO:3 corresponds to the amino acid sequence of the IFN-α analog IFN-α-N7, where amino acid residues 19-27 of mature human IFN-α are substituted with amino acids 19-27 of mature ovine IFN-τ.

### DETAILED DESCRIPTION

### I. Definitions

“Interferon-tau”, abbreviated as IFN-τ or interferon-τ, refers to any one of the Type I interferon proteins identified in the literature as interferon-tau. See, e.g., Ott, et al., J. Interferon Res., 11:357 (1991); Helmer, et al., J. Reprod. Fert., 79:83 (1987); Imakawa, et al., Mol. Endocrinol, 3:127 (1989); Whaley, et al., J. Biol. Chem., 269:10846 (1994); Bazer, et al., WO 94/10313 (1994). IFN-τ sequences have been identified in various ruminant species, including but not limited to, cow (Bovine sp., Helmer, S. D., J. Reprod. Fert., 79:83 (1987); Imakawa, K., Mol. Endocrinol., 119:532 (1988)), sheep (Ovine sp.), musk ox (Ovibos sp.), giraffe (Giraffa sp., GenBank Accession no. U55050), horse (Equus caballus), zebra (Equus burchelli, GenBank Accession no. NC005027), hippopotamus (Hippopotamus sp.), elephant (Loxodonta sp.), llama (Llama glama), goat (Capra sp., GenBank Accession nos. AY357336, AY357335, AY347334, AY357333, AY357332, AY357331, AY357330, AY357329, AY357328, AY357327), and deer (Cervidae sp.). The nucleotide sequences of IFN-τ for many of these species are reported in public databases and/or in the literature (see, for example, Roberts, R. M. et al., J. Interferon and Cytokine Res., 18:805 (1998), Leaman D. W. et al., J. Interferon Res., 12:1 (1993), Ryan, A. M. et al., Anim. Genet., 34:9 (1996)). The term “interferon-tau” intends to encompass the interferon-τ protein from any ruminant species, exemplified by those recited above, that has at least one characteristic from each of the two groups of characteristics listed above.

“Ovine IFN-τ”, also abbreviated as OvIFN-τ, refers to a protein having the amino acid sequence as identified herein as SEQ ID NO:1, and to proteins having amino acid substitutions and alterations such as neutral amino acid substitutions that do not significantly affect the activity of the protein, such as the IFN-τ protein identified herein as SEQ ID NO:2. More generally, an ovine IFN-τ protein is one having about 80%, more preferably 90%, sequence homology to the sequence identified as SEQ ID NO:1. Sequence homology is determined, for example, by a strict amino acid comparison or using one of the many programs commercially available, for example, the LALIGN program with default parameters. This program is found in the FASTA version 1.7 suite of sequence comparison programs (Pearson and Lipman, PNAS, 85:2444 (1988); Pearson, Methods in Enzymology, 183:63 (1990); program available from William R. Pearson, Department of Biological Chemistry, Box 440, Jordan Hall, Charlottesville, Va.).

“Variant IFN” proteins refers to two classes of proteins: (1) “hybrid interferon/interferon-tau proteins”, also abbreviated as hybrid INF/IFN-τ proteins, or simply INF/IFN-τ proteins, also known as chimeric proteins (N-terminal fusion proteins) disclosed by Villarete et al. in U.S. Patent Publication No. 2003/0130486, and U.S. Pat. Nos. 5,939,286 and 6,174,996 (both to Johnson et al.), and (2) substitutional “analogs” disclosed in U.S. Pat. No. 6,204,022 (Johnson et al.), which are all herein incorporated by reference in their entirety. The proteins contemplated for use in the subject methods, including hybrid INF/IFN-τ proteins and substantially homologous analogs of INF-α, are functionally equivalent to a native INF protein. An analog is substantially homologous to the native protein when at least about 80%, more preferably about 90%, and most preferably at least about 95% of its amino acid sequence is identical to the amino acid sequence of the native protein. An analog may differ by as few as 1, 2, 3, or 4 amino acids. A variant protein is functionally equivalent when it possesses substantially the same biological activity as the native IFN. Note that functionally equivalent variants may differ substantially in sequence, and these variants are also within the scope of the invention.

A condition, disease, illness or infection “responsive to interferon therapy” is one in which the existence, progression, or symptoms of the condition, disease, illness or infection is altered upon administration of an interferon, in particular a Type-I interferon, and more particularly, IFN-τ or variant IFN proteins. More preferably, a condition, disease, illness or infection responsive to interferon therapy is one where the existence, progression, or symptoms of the condition, disease, illness or infection are alleviated by IFN-τ or variant IFN proteins.

“Treating” a condition, disease, illness or infection refers to administering a therapeutic substance effective to reduce the symptoms of the condition, disease, illness or infection and/or lessen the severity of the condition, disease, illness or infection.

“Respiratory tract” is defined as the system of body comprising the nose, nostrils, nasal passages, nasal cavity, pharynx and larynx, which are sometimes collectively referred to as the upper respiratory tract, and the trachea, brochi, bronchioles, alveoli and their clusters, the alveolar sacs, which make up the lungs and are sometimes collectively referred to as the lower respiratory tract. The various parts/tissues of the respiratory system, alone or in combination with other parts/tissues, may be called “regions.”

### II. Therapeutic Formulations

A. Therapeutic Interferons

In one aspect, a treatment method comprising administering an interferon protein to the respiratory tract is provided, for treating systemic disorders that are responsive to interferon. In one embodiment the interferon protein a Type I interferon, such as interferon-alpha (IFN-τ-α), interferon-omega (IFN-Ω), or IFN-τ, and more particularly, an ovine IFN-τ. In another embodiment, the interferon is a Type II interferon, such as interferon-beta.

In a preferred embodiment, the interferon therapeutic agent is IFN-τ or a variant IFN-τ. Relative to other interferons, ovine IFN-τ shares about 45-55% identity with IFN-αs from human, mouse, rat, and pig and 70% homology with bovine IFN-αII, now referred to as IFN-Ω. A cDNA of ovine IFN-τ and several cDNA sequences which may represent different isoforms have been reported in the literature (Imakawa, K. et al, Nature, 330:377-379, (1987); Stewart, H. J., et al, Mol. Endocrinol. 2:65 (1989); Klemann, S. W., et al., Nuc. Acids Res. 18:6724 (1990); and Charlier, M., et al., Mol. Cell. Endocrinol. 76:161-171 (1991)). All are approximately 1 kb with a 585 base open reading frame that codes for a 23 amino acid leader sequence and a 172 amino acid mature protein.

The 172 amino acid sequence of ovine-IFN-τ is set forth, for example, in U.S. Pat. No. 5,958,402. In another embodiment, the therapeutic agent is bovine-IFN-τ, the sequence of which is described, for example, in Helmer et al., J. Reprod. Fert., 79:83-91 (1987) and Imakawa, K. et al., Mol. Endocrinol., 3:127 (1989). The sequences of ovine-IFN-τ and bovine-IFN-τ from these references are hereby incorporated by reference. An amino acid sequence of ovine IFN-τ is shown herein as SEQ ID NO:1. A modified amino acid sequence of ovine IFN-τ is shown herein as SEQ ID NO:2.

Methods for the recombinant production of IFN-τ is described in both the scientific literature (Ott, et al., J. Interferon Cytokine Res., 11:357-364 (1991); Soos, J. M. et al., J. Immunol., 155:2747 (1995)) and the patent literature (WO/94/10313; US 2003/0049277), and these documents are herein incorporated by reference in their entirety.

Other embodiments of the therapeutic agent include a polypeptide analog of a native human IFN-α protein, wherein the sequence of amino acids in the analog that corresponds to the sequence of residues 1-27 of the native IFN-α differs therefore at one or more of positions 19, 20, 22, 24, and 27, provided that the sequence in the analog does not differ from the corresponding native IFN-α only by the presence of Ser, Thr, As, Gln, or Gly at the amino acid residue corresponding to position 22. For example, one preferred embodiment is listed as SEQ ID NO:3, in which the modified sequence differs from the native sequence by substitutions at positions 19, 20, 22, 24, and 27. The analog is capable of exhibiting lower toxicity relative to the native IFN-α in, for example, an assay involving human mononuclear cells in culture. Such analog proteins are fully disclosed in U.S. Pat. No. 6,204,022, issued to Johnson et al., which is incorporated herein by reference.

In another embodiment, the therapeutic agent is a fusion protein comprised of an IFN-α protein portion and an IFN-τ protein portion. As will now be described, an IFN-α, such as IFN-αD, can be modified at either the N-terminus or the C-terminus with a contiguous segment of amino acid residues, having any length, from an IFN-τ. The terms fusion and hybrid are used herein to intend the concept of joining a segment of a first protein to a segment of a second protein to form a new hybrid or fusion protein. These hybrid or fusion proteins fall within the more general term of an IFN-α analog protein. It is also further contemplated that analog sequences can be constructed from these fusion or hybrid protein in a manner consistent with the structures herein defined, as well as more complex chimeras containing more than two discrete regions derived from interferon proteins.

A fusion protein comprised of a first segment that contains the N-terminal amino acid sequence of an interferon-τ protein and a second segment that contains the C-terminal amino acid sequence of a non-τ interferon Type I polypeptide is contemplated. The two segments are joined in the region of a mature interferon polypeptide between about residues 8 and 37. In another general embodiment, the two segments are spliced in a region corresponding to the portion of a mature interferon polypeptide between about residues 8 and 28. In yet another general embodiment, the two segments are spliced in a region corresponding to the portion of a mature interferon polypeptide between about residues 8 and 22. In still another general embodiment, the two segments are spliced in a region corresponding to the portion of a mature interferon polypeptide between about residues 8 and 16. As mentioned above, and as described in U.S. Pat. No. 5,939,286, which is incorporated by reference herein, the N-terminal region of IFN-τ confers decreased toxicity to IFN-α, therefore the N-terminal region of IFN-τ is particularly attractive to use in a fusion or hybrid protein.

In yet another embodiment, the therapeutic agent as provided in the any of the above embodiments may be prepared as a conjugate with hydrophilic polymer compounds, as disclosed in U.S. Provisional Patent Application No. 60/692,484, filed Jun. 20, 2005. The conjugate is preferably a covalent conjugate, of one or more, or any combination of synthetic or naturally occurring polymers, oligomers or small molecules. The desired characteristics of the modified protein include, without limitation, increased circulation time in the body, reduced immunogenicity and retention of substantial therapeutic activity.

B. Liquid and Fine Power Preparations of the Therapeutic Protein

The therapeutic agent can be delivered as a liquid or a dry powder. Either may be in aerosol form or non-aerosol form. Non-aerosol formulations for inhalation may take the form of a liquid that is typically, but not necessarily, an aqueous suspension. Formulations may be used as well, in which the carrier is typically a sodium chloride solution having a concentration that is isotonic relative to normal body fluid. In addition to the carrier, the liquid formulations may include water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof).

Dry powder formulations may also be used in non-aerosol formulations for inhalation, particularly insufflations in which the powder has an average particle size of about 0.1 μm to 50 μm, with a preferred size range of 1 μm to about 25 μm. Descriptions of aerosols are in U.S. Pat. No. 6,403,597 and U.S. Pat. No. 6,905,701, which are incorporated herein by reference.

Aerosol formulations are contemplated for delivery to the nasal mucosa, particularly in the posterior of the nasal passages and cavity, and the “deep” lung tissue, via for example the alveoli. Generally speaking, there are two classifications of liquid aerosols: a solution aerosol in which the therapeutic agent is solubilized in a carrier, or a dispersive aerosol in which the therapeutic agent is suspended or dispersed throughout a carrier and an optional solvent. The term “aerosol” includes any suspended phase of the therapeutic agent that is gas-borne and is capable of being inhaled into the lower (bronchi, bronchioles, alveoli, etc.) or upper (nasal passages, etc.) respiratory tract. The suspended phase may either be a liquid or a solid.

Whether the IFN-τ based therapeutic agent is provided as a solution or a microfine powder, an atomizer may be provided to insure a sufficient reduction in particle size so that inhalation of the composition from the nasal or oral cavity is effective to deliver the drug to the desired target region with the respiratory tract.

A liquid aerosol form includes a gas-borne suspension of droplets of the compounds of the instant invention, which may be produced in a metered dose inhaler or nebulizer, or in a mist sprayer. A solid aerosol form includes a dry powder composition of the therapeutic agent suspended in air or other carrier gas. The powder suspended in gas may be delivered by insufflation from an inhaler device, for example.

The preferred range of concentration of the therapeutic agent in solutions for making aerosols is 0.1-100 milligrams (mg)/milliliter (mL), more preferably 0.1-30 mg/mL, and most preferably, 1-10 mg/mL. Aerosol solutions are usually, but not necessarily, buffered with a physiologically compatible buffer. Exemplary buffers include phosphate or bicarbonate. The usual pH range of the aerosol solution is 5 to 9, preferably 6.5 to 7.8, and more preferably 7.0 to 7.6. Typically, sodium chloride is added to adjust the osmolarity to the physiological range, which is preferably within 10% of isotonic. Formulation of such solutions for creating aerosol inhalants is discussed in Remington's Pharmaceutical Sciences, see also, Ganderton and Iones, Drug Delivery to the Respiratory Tract, Ellis Horwood (1987); Gonda, Critical Reviews in Therapeutic Drug Carrier Systems 6:273-313 (1990); and Raeburn et al., J. Pharmacol. Toxicol. Methods 27:143-159 (1992).

When the therapeutic agent to be used is a liquid-based formulation, preferred formulations have a tonicity which does not induce an irreversible change in the mucosa in the respiratory tract, or at least do not induce a change at the targeted region. The tonicity of the solution, expressed as a ratio to the tonicity of a 0.9% (w/v) physiological saline solution, is within the range 0.33 to 3, preferably 0.5 to 2, and most desirably 0.75 to 1.7.

Any of the known means commonly used to make aerosol inhalant pharmaceuticals can be used to convert solutions of the therapeutic agent into aerosols. Such methods generally comprise pressurizing or providing a means of pressurizing a container of the solution, usually with an inert carrier gas. The pressurized gas is then passed through a small orifice to dispense droplets of the solution into the mouth and trachea of the animal to which the drug is to be administered. A mouthpiece is typically fitted to the outlet of the orifice to facilitate delivery into the mouth and trachea.

C. Swellable Polymers and Adhesives

The therapeutic agent can also be released from swellable polymers or adhesives configured for application to the nasal cavity. Generally, adhesives for drug delivery consist of a matrix of a hydrophilic, e.g., water soluble or swellable, polymer or mixture of polymers which can adhere to a wet mucous surface. These adhesives may be formulated as ointments, thin films, and other forms. Some of these adhesives have been formulated to permit adsorption through the mucosa into the circulatory system of the individual. Adhesives are described in U.S. Pat. No. 5,516,523, which is incorporated by reference herein.

While polymers generally function as a pharmaceutical base, it can be advantageous to add a different commonly used base in accordance with the particular dosage form to be manufactured. For example, macrogels, propylene glycol, glycerin, etc. can be used where necessary.

The therapeutic composition may optionally contain a hydrophilic low molecular weight compound to assist deliver of the protein agent to the cells lining the tract. An example of such a compound is described in U.S. Pat. No. 5,725,852, which is incorporated by reference herein. The water-soluble physiologically active peptide or protein may diffuse from the base to the mucosal surface through the continuous passages created by the hydrophilic low molecular-weight compound.

The hydrophilic low molecular weight compound can be any such compound that absorbs moisture from the mucosa or the atmosphere and dissolves the therapeutic agent (which is water-soluble). The molecular weight of said hydrophilic low molecular weight compound is not more than 10 kD and preferably not more than 3 kD. The compound may be a polyol compound such as glycerin or polyethylene glycol (average molecular weight of 2-3 kD). The compound may also be an oligosaccharide, a disaccharide, or a monosaccharide such as sucrose, mannitol, lactose, L-arabinose, D-erythrose, D-ribose, D-xylose, D-mannose, D-galactose, lactulose, cellobiose, gentibiose, etc. Other examples of the hydrophilic low molecular weight compound include N-methylpyrrolidone and alcohols (e.g. oligovinyl alcohol, ethanol, ethylene glycol, and propylene glycol). These hydrophilic low molecular weight compounds are used alone or in combination.

The base for the therapeutic composition of the present invention may be a hydrophilic compound having a capacity to disperse the protein and additives. The molecular weight of such hydrophilic compound is at least 1 kD, preferably at least 10 kD, and more preferably at least 100 kD. The compound need only be a pharmaceutically acceptable substance. Exemplary compounds include, but are not limited to, polycarboxylic acids or salts thereof or carboxylic anhydrides (e.g. maleic anhydride) with other monomers (e.g. methyl(meth)acrylate, acrylic acid, etc.); hydrophilic vinyl polymers such as polyvinyl acetate, polyvinyl alcohol, polyvinylpyrrolidone, etc.; cellulose derivatives such as hydroxymethylcellulose, hydroxypropylcellulose, etc.; and natural polymers such as chitosan, collagen, sodium alginate, gelatin, hyaluronic acid, etc. and nontoxic metal salts thereof; and synthetic fatty acid esters such as polyglycerin fatty acid esters, sucrose fatty acid esters.

These hydrophilic polymers can be used alone or in combination and a necessary structural integrity can be imparted by partial crystallization, ionic bonding, crosslinking or the like. Any of these hydrophilic polymers may be molded into a film form, microparticles (e.g. microspheres) or nanoparticles (e.g. nanospheres) and applied to the nasal mucosa. They may also be applied in powdery form or as a solution to the mucosa. The solution may or may not be viscous. As further disclosed, other additives may be included in the composition, such as for promoting absorption, enhancing residence times or inhibiting enzymes.

A biodegradable synthetic polymer can be used as a base with the hydrophilic polymer. The biodegradable polymer typically includes, but is not limited to, polylactic acid, poly(lactic acid-glycolic acid) copolymer, polyhydroxybutyric acid, poly(hydroxybutyric acid-glycolic acid) copolymer and mixtures thereof. These biodegradable polymers may be molded into a form as described above.

In one embodiment, IFN-τ is dispersed in a base. Interferon-τ may be released from the base through diffusion, disintegration of the base in the case of it being a biodegradable polymer, or by the formation of water channels in the base. Release may be further promoted by forming the base from a polymer having a glass transition temperature similar to (within 5° C. of body temperature) or lower than the body temperature. In such a case, the polymer base softens when applied to the body, which then accelerates diffusion of the therapeutic agent out of the base.

In another embodiment, the therapeutic agent (e.g. IFN-τ and the like) is loaded within microcapsules or nanocapsules prepared from a polymer. The microcapsules or nanocapsules are then dispersed in a biocompatible dispersing medium that is applied to the mucosal tissues. Encapsulation prevents the therapeutic agent from coming into direct contact with the cilia, as well as degradative enzymes present within the mucosal layer. In preferred embodiments, the capsules may be further formulated to provide absorption enhancement and/or mucosal adhesion. One means of enhancing absorption of the therapeutic agent is associated with calcium sequestration. Compounds such as glycofurolam, cyclodextrins or L-α-lysophosphatylcholines may be used to induce greater absoption. Mucosal adhesives are useful to promote longer residence time in the nasal passages. Exemplary excipients useful as an adhesive include starches, gelatin, chitosan, or dextran.

D. Therapeutic Compositions

Therapeutic interferon compositions for delivery to the respiratory tract may further comprise pharmaceutically acceptable additives such as, for example, stabilizers, preservatives, surfactants, viscosity enhancers, pH control agents (buffering agents), solubilizers. Several of these have been described above in connection with liquid preparations of the therapeutic agent.

In one preferred embodiment, whether administration employs a liquid, solid powder, swellable polymer, adhesive or ointment format, the composition contains a stabilizer, such as histidine, which acts to stabilize the tertiary structure of IFN-τ and to improve solubility in aqueous formulations, as described in WO 2004/032863, which is incorporated by reference herein. Other exemplary stabilizers include serum albumin.

The composition is provided in a pH range that does not substantially affect the activity of the physiologically active peptide or protein and is physiologically acceptable. The preferred range is about pH 5 to about pH 7. The more preferred range is about pH 5.5 to about pH 6.5. A guiding operative indicator when choosing the pH, particular when the targeted region is the upper respiratory tract, is to not irritate the mucosal lining of the nasal passages. Accordingly, the optimal pH needed to avoid irritation may vary for a particular application or patient group (e.g. children vs. adult), and can be ascertained by observing whether the cilial beat frequency is perturbed using microscopy or histopathological means depending on the particular protein therapeutic agent used, an acidic or basic solution is preferred to a neutral solution for enhanced stability and/or absorption of the peptide.

Because a variety of proteolytic enzymes are present in the mucosa of the respiratory tract and in the environment the drug is administered, compositions may further comprise protease inhibitors or peptidase inhibitors to block degradation of administered protein therapeutic agent and thereby ensure better delivery and bioavailability. Exemplary protease inhibitors include but are not limited to gabaxate mesylate, α1-antitrypsin, aprotinin, leupepsin, α2-macroglobulin, pepstatin and egg white or soybean trypsin inhibitor. These inhibitors can be used alone or in combination. The protease inhibitor may be incorporated in the hydrophilic polymer, coated on the surface of the dosage form which is to contact the mucosa or incorporated in the superficial phase.

An embodiment of the therapeutic composition may be further supplemented with an absorption promoter that assists in the absorption and diffusion of the therapeutic protein. The absorption promoter may be any promoter that is pharmaceutically acceptable. Delivery-enhancing transporter compounds are described in U.S. Pat. No. 6,759,387, which is incorporated herein by reference. The enhanced penetration of the conjugated agents into and across one or more layers of the epithelial tissue that is provided by the delivery-enhancing transporters of the invention results in amplification of the advantages that respiratory tract delivery has over other delivery methods. For example, rapid onset of pharmacological activity can result from respiratory tract administration. Lower doses of an agent will often yield the desired effect, a local therapeutic effect can occur rapidly and systemic therapeutic blood levels of the agent are obtained quickly. Moreover, respiratory tract administration generally has relatively few side effects.

Exemplary absorption promoters include, but are not limited to, sodium salicylate and salicylic acid derivatives (acetyl salicylate, choline salicylate, salicylamide, etc.), amino acids and salts thereof (e.g. monoaminocarboxlic acids such as glycine, alanine, phenylalanine, proline, hydroxyproline, etc., hydroxyamino acids such as serine etc., acidic amino acids such as aspartic acid, glutamic acid, etc. and basic amino acids such as lysine etc., inclusive of their alkali metal or alkaline earth metal salts), N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts), substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters and, as mentioned earlier, glycofurolam, cyclodextrins or L-α-lysophosphatylcholines. Any pharmaceutically acceptable base or vehicle can be used to disperse the absorption additive.

Cytidine nucleotide derivatives can facilitate protein absorption by mucosal tissues and are described in U.S. Pat. No. 5,482,706, which is incorporated by reference herein. To prepare such solutions, a solution of IFN-τ in water or physiological saline where the cytidine nucleotide derivative is added or a solution of a vacuum-dried or freeze-dried preparation containing both the physiologically active peptide or protein and the cytidine nucleotide derivative in water or physiological saline can be sprayed from a nebulizer or other suitable spray means. It is also possible to add sodium alginate, sodium hyaluronate, hydroxypropylcellulose, other cellulose derivatives and the like to the solution to make it viscous so as to prolong the local residence time of the composition.

In the manufacture of a therapeutic composition comprising a cytidine derivative, the cytidine nucleotide derivative may be directly blended with IFN-τ. Alternatively, the cytidine nucleotide derivative may be hydrophilic, biodegradable or other polymer and then blended with IFN-τ. In certain instances, the derivative may be coated on the surface of a dosage form which is to contact the mucosa. All that is necessary is that the therapeutic protein and the cytidine nucleotide derivative should be concurrently brought into contact with the mucosa.

The therapeutic composition of an embodiment may be stored at room temperature or preferably in the cold before use. Contamination must be prevented when a multidose unit is used to administer the therapy. An exemplary way of preventing contamination includes a device for precluding the backflow of the body fluid into the dispensing container, preferably in combination with storage in the cold after each dispensing. To prevent growth of adventitious microorganisms within the container, a pharmaceutically acceptable preservative or/and an antimicrobial agent may be added to the composition.

The amount of interferon included in the compositions of any embodiment is designed to be a quantity that provides an effective amount of the protein agent needed to reduce the symptoms of the condition, disease, illness or infection and/or lessen the severity of the condition, disease, illness or infection under treatment.

The amount of IFN-τ or variant IFN contained in the therapeutic composition is be adjusted according to, without limitation, the activity of the particular substance, the desired dose, the efficiency of the delivery, the format of administration, and the targeted region within the respiratory tract. Because administered proteins are not completely absorbed, due in substantial part to the factors cited above, it is preferable to estimate the amount needed to be administered in order to arrive at the desired dosage within the body. Furthermore, the dosage can vary depending on the age, gender and body weight of the recipient.

### III. Treatment Methods—Systemic and Local

A. Use of the Preparations

The therapeutic compositions, in any of the various embodiments described, are administered to the respiratory tract. According to the nature of the disease, condition, illness or infection to be treated, the dosage, the medically preferred site and in some instances the patient preference, the mode of administration, that is, the format for administering the therapeutic dose can be selected.

Respiratory tract administration is useful for both treatment of pulmonary conditions and for delivery of drugs to distant target organs via the circulatory system. Many such drugs and diagnostic agents can be administered through the respiratory tract by, for example, conjugation to the delivery-enhancing transporters described in U.S. Pat. No. 6,759,387.

The following are some typical modes of administration, although these are by no means exclusive choices. Nasal drug delivery can be carried out using unit dose containers, pump sprays, droppers, squeeze bottles, airless and preservative-free sprays, compressed air nebulizers, metered dose inhalers, and pressurized metered dose inhalers. It is important that the delivery device protect the drug from contamination and chemical degradation. The device should also avoid leaching or absorption as well as provide an appropriate environment for storage.

Hydrophilic polymers used in mucosal delivery of IFN-τ may be molded into e.g., a film form and applied to the nasal mucosa. These polymers may also be molded into microspheres, nanospheres, or the like, and applied to the mucosa of the nose. The hydrophilic polymers may also be applied in powdery form or as a solution, which may be viscous, to the mucosa. It may also be advantageous to combine a mucosal adhesive with a microcapsule or microsphere to promote longer residence time in the body. The capsule or sphere may either be comprised of the adhesive material, or another base polymer may be coated with the adhesive.

It will be appreciated that the IFN-τ or variant IFN preparation can be administered alone or in combination with another therapeutic agent, given by any route of administration. Selection of a second agent is made by a primary caregiver and typically relates to the condition from which the patient suffers. By way of example, IFN-τ can be administered to the mucosal tissue for treatment of a systemic autoimmune condition, such as multiple sclerosis, along with a second drug, such as azathioprine, cyclophosphamide, corticosteroids (prednisone, prednisolone, others), cyclosporine, mycophenolate mofetil, antithymocyte globulin, muromonab-CD3 monoclonal antibody, mercaptopurine, mitoxantrone, glatiramer acetate (Copaxone), interferon-beta (Avonex™, Betaseron™, Ribif™), daclizumab, methotrexate, sirolimus, tacrolimus, and others. The second drug can be administered orally, parenterally, or topically. Selection of other second therapeutic agents that might be used in combination with IFN-τ or variant IFN to treat any of a variety of conditions are readily identified by trained medical personnel.

B. Conditions for Treatment

Diseases which may be treated using methods of the present invention include autoimmune, inflammatory, proliferative and hyperproliferative diseases, as well as cutaneous manifestations of immunologically mediated diseases. Interferon-like agents can be used as an effective therapeutic agent to treat diseases with an immunopathologic basis. Such diseases are characterized by inadequate either an immune response and persistence of the disease or by an apparent hyperactive immune response that can degenerate tissue from inflammatory conditions and related physical manifestations. In addition, various infections and illnesses, particularly those caused by viruses can be treated by methods of the present invention owing to the anti-viral properties possessed by INF-τ and variant IFN. Further descriptions of the conditions treated by the subject method and the operation of the method follow.

C. Systemic Treatment of Viral Infections (e.g. Hepatitis C)

The method of the invention may be used to treat viral infections and diseases wherein administration via the respiratory tract is used to provide a systemic dose of the therapeutic agent. As noted above, the antiviral activity of IFN-τ and variant IFN have broad therapeutic application without the toxic effects that are usually associated with IFN-α, and IFN-τ exerts is therapeutic activity without adverse effects on the cells. The relative lack of cytotoxicity of IFN-τ makes it extremely valuable as an in vivo therapeutic agent and sets IFN-τ apart from most other known antiviral agents and all other known interferons.

Formulations containing IFN-τ or variant IFN can be administered to the upper or lower respiratory tract for systemic delivery to inhibit viral replication. For use in treating a viral infection, the protein is administered at a dose sufficient to achieve a measurable increase in blood IL-10 in the patient. Thereafter, treatment is continued at an effective dose, independent of further changes in blood IL-10 levels, for example, a fall in IL-10 blood levels due to reduction in viral load. Administration of IFN-τ is continued until the level of viral infection, as measured for example from a blood viral titer or from clinical observations of symptoms associated with the viral infection, abates.

Other viral infections or diseases may be treated likewise. The viral infection can be due to an RNA virus or a DNA virus. Examples of specific viral diseases which may be treated by administration of IFN-τ or variant IFN include, but are not limited to, hepatitis A, hepatitis B, hepatitis C, non-A, non-B, non-C hepatitis, Epstein-Barr viral infection, HIV infection, herpes virus (EB, CML, herpes simplex), human papilloma virus, poxvirus, picorna virus, adenovirus, rhino virus, HTLV I, HTLV II, and human rotavirus. The patient may be co-treated during the therapeutic agent treatment period with a second antiviral agent such as are known in the art or are shown to effective in treating such viral infections.

D. Diseases Localized to the Respiratory Tract

The methods of the invention are also useful for the treatment of disease, conditions, illnesses and infections localized to the respiratory tract, such as the lungs or the nasal cavity. Exemplary conditions treated by the therapeutic agents and methods of delivery include chronic inflammatory lung diseases, such as for example, asthma, pneumoconiosis, chronic obstructive pulmonary disease, nasal polyps and pulmonary fibrosis. Typically such diseases are characterized by an invasive inflammatory process, and thickening of the affected tissues.

Also contemplated is the treatment of Idiopathic pulmonary fibrosis (IPF) is a chronic progressive interstitial lung disease of unknown etiology, characterized by inflammation and fibrosis of the lung parenchyma. Treatment with immunosuppressants such as the therapeutic agents of the subject invention, alone or in combination with systemic corticosteroids may benefit patients with lung histopathology patterns of high cellular inflammation and less fibrosis. Although a systemic disease, lymphomatoid granulomatosis, a rare angiodestructive lymphoproliferative disease of unknown etiology, is characterized by prominent pulmonary involvement and may also be treated by the therapeutic agents of the subject invention. Also, in some cases following lung transplant surgery administration of interferon-τ and the like are useful to treat viral infections such as EPV, one of the primary risk factors following surgery.

Viral infections are common in the upper respiratory tract, and inflammatory responses caused by these viral may also be treated by the methods and therapeutic agents of the subject invention. Infections by rhinovirus, adenovirus, Epstein-Barr virus, herpes simplex virus, influenza, parainfluenza, coronavirus, enterovirus, respiratory syncytial virus, cytomegalovirus and human immunodeficiency virus are indicated for treatment.

Formulations containing IFN-τ or variant IFN can be administered to the upper respiratory tract for local delivery to inhibit viral replication. For treating the viral infection, the protein is administered via the disclosed methods, such as spray, drops, aerosol, ointment, dry powder or adhesive, for example, at a dose sufficient to achieve a measurable response in the patient.

E. Hyperproliferation of Cells

In another embodiment, the methods of the invention are contemplated for treatment of conditions characterized by cell hyperproliferation. IFN-τ exhibits potent anti-proliferation activity. Accordingly, a method of inhibiting cellular growth by administering IFN-τ or variant IFN to the respiratory tract is contemplated, in order to inhibit, prevent, or slow uncontrolled cell growth.

Examples of cell proliferation disorders in humans which may be treated by nasally-administered IFN-τ include, but are not limited to, lung large cell carcinoma, colon adenocarcinoma, promyelocytic leukemia, T cell lymphoma, cutaneous T cell lymphoma, breast adenocarcinoma, steroid sensitive tumors, hairy cell leukemia, Kaposi's Sarcoma, chronic myelogenous leukemia, multiple myeloma, superficial bladder cancer, ovarian cancer, mesothelioma, Burkitt lymphoma, malignant lymphoma, nasopharyngeal carcinoma, and glioma.

For use in treating a cell proliferation condition, for example, IFN-τ is administered in a dose sufficient manner to achieve an initial measurable increase in blood IL-10 in a patient. Thereafter, treatment is continued at an effective dose, independent of further changes in blood IL-10 levels, for example, a fall in IL-10 blood levels due to a reduction in cancer cells in the body. Administration of IFN-τ at an effective dose is continued until a desired level of regression is observed, as measured for example, by tumor size or extent of cancer cells in particular tissues. The patient may be co-treated during the IFN-τ treatment period with a second anticancer agent, e.g., cis-platin, doxorubicin, or taxol by the appropriate route of administration.

F. Autoimmune Conditions (Multiple Sclerosis, Psoriasis)

Mammalian immune systems must be able to distinguish between “self” and “non-self” antigens in order to successfully defend against microorganisms with minimal harm to the mammal. “Non-self” antigens are selectively recognized in a normal mammalian immune system. Autoimmune disorders involve recognition and a subsequent immune response against “self” antigens that can damage tissues in the mammal. Autoimmune diseases are described in U.S. Pat. Nos. 5,882,640, 6,204,022, and 6,861,056, which are incorporated by reference herein, and the application of IFN-τ is described in U.S. Pat. Nos. 6,060,450 and 5,906,816, which are incorporated herein by reference.

A variety of factors can lead to an autoimmune disease, including genetic factors and exogeneous factors. Cross-reactivity between a “non-self” antigen and a similar “self” antigen can lead to the development of an autoimmune reaction. For example, tissues in the eye such as the lens and the cornea that are not normally exposed to lymphocytes can be recognized as “non-self” by the immune system when lymphocyte contact does take place. There is suggestion that autoimmune diseases can develop from a disruption in synthesis of interferons and other cytokines. (Theofilopoulos et al., Annu. Rev. Immunol., 23:307-336, 2005).

Autoimmune disorders may be loosely grouped into those primarily restricted to specific organs or tissues and those that affect the entire body. Examples of organ-specific disorders (with the organ affected) include multiple sclerosis (myelin coating on nerve processes), type I diabetes mellitus (pancreas), Hashimoto's thyroiditis (thyroid gland), pernicious anemia (stomach), Addison's disease (adrenal glands), myasthenia gravis (acetylcholine receptors at neuromuscular junction), rheumatoid arthritis (joint lining), uveitis (eye), psoriasis (skin), Guillain-Barre Syndrome (nerve cells) and Grave's disease (thyroid). Systemic autoimmune diseases include systemic lupus erythematosus and dermatomyositis.

Autoimmune diseases particularly amenable for treatment using the methods of the present invention include multiple sclerosis, type I (insulin dependent) diabetes mellitus, lupus erythematosus, amyotrophic lateral sclerosis, Crohn's disease, rheumatoid arthritis, stomatitis, asthma, uveitis, allergies and psoriasis. IFN-τ is a preferred therapeutic agent in accordance with this embodiment. Some data have indicated better efficacy, i.e., a more pronounced immunomodulatory effect, where the interferon is not homologous to the species being treated. IFN-τ can be administered alone or in combination with interferon-alpha, interferon-beta, or interferon-gamma.

The method of the present embodiment is used to therapeutically treat and thereby alleviate autoimmune disorders, such as those discussed above. Treatment of an autoimmune disorder is exemplified herein with respect to the treatment of EAE, an animal model for multiple sclerosis. When used to treat an autoimmune disorder, IFN-τ or variant IFN is administered at a dose sufficient to achieve the measurable increase in IL-10 during the initial phase(s) of administration. Once a desired effective dose is achieved, the patient is treated over an extended period with an effective therapeutic dose, independent of further changes in IL-10 blood levels. The treatment period extends at least over the period of time when the patient is symptomatic. Upon cessation of symptoms associated with the autoimmune condition, the dosage may be adjusted downward or treatment may cease. The patient may be co-treated during the treatment period of treatment with another agent, such as a known anti-inflammatory or immune-suppressive agent by similar or different routes of administration.

Also contemplated is a method of preventing progression of an autoimmune condition, by administering the IFN-τ or variant IFN in a dose that elevates the IL-10 level in a subject. Also contemplated is a method of inhibiting onset of an autoimmune condition, by administering a dose effective to increase IL-10 serum levels, preferably with no change or a reduction in the interferon-gamma level. Also contemplated is a method of treating an autoimmune condition by administering a dose effective to increase the IL-10/IL-12 serum ratio. As discussed above, the dose is provided in amounts typically greater than about 5×10⁸ units/day.

Psoriasis, a skin disorder in which lesions form, is now thought to be an autoimmune disease mediated by T-lymphocytes. (Krueger, J. G. and Bowcock, A., Ann. Rheum. Dis., 64:Suppl2:230-36, 2005). Psoriasis has been treated with ultraviolet light and oral methotrexate. However, both of these therapies involve considerable toxicity. An embodiment includes treatment of psoriasis by respiratory tract administration of IFN-τ. The preferred range of dosage of IFN-τ is 10³-10⁵ IU.

G. Hyperimmune Reactions

Hypersensitivity reactions often arise in the course of tissue and organ transplantation. Rejection of a transplant is an organism's normal reaction to invading foreign antigens. In particular, transplantation of tissues or organs such as the eye, which is not normally exposed to lymphocytes, skin, heart, kidney, liver, bone marrow, and other organs, have a high rate of rejection.

Other examples beside transplant rejection of hypersensitivity disorders include asthma, eczema, atopical dermatitis, contact dermatitis, other eczematous dermatitides, seborrheic dermatitis, rhinitis, Lichen planus, Pemplugus, bullous Pemphigoid, Epidermolysis bullosa, uritcaris, angioedemas, vasculitides, erythemas, cutaneous eosinophilias, Alopecia greata, atherosclerosis, primary biliary cirrhosis and nephrotic syndrome. Related diseases include intestinal inflammations, such as Coeliac disease, proctitis, eosinophilia gastroenteritis, mastocytosis, inflammatory bowel disease, Crohn's disease and ulcerative colitis, as well as food-related allergies.

The methods described herein are advantageous for treating conditions relating to immune system hypersensitivity. There are four types of immune system hypersensitivity. Type I, or immediate/anaphylactic hypersensitivity, is due to mast cell degranulation in response to an allergen (e.g., pollen), and includes asthma, allergic rhinitis (hay fever), urticaria (hives), anaphylactic shock, and other illnesses of an allergic nature. Type II, or autoimmune hypersensitivity, is due to antibodies that are directed against perceived “antigens” on the body's own cells. Type III hypersensitivity is due to the formation of antigen/antibody immune complexes which lodge in various tissues and activate further immune responses, and is responsible for conditions such as serum sickness, allergic alveolitis, and the large swellings that sometimes form after booster vaccinations. Type IV hypersensitivity is due to the release of lymphokines from sensitized T-cells, which results in an inflammatory reaction. Examples include contact dermatitis, the rash of measles, and “allergic” reactions to certain drugs.

The mechanisms by which certain conditions may result in hypersensitivity in some individuals are generally not well understood, but may involve both genetic and extrinsic factors. For example, bacteria, viruses or drugs may play a role in triggering an autoimmune response in an individual who already has a genetic predisposition to the autoimmune disorder. It has been suggested that the incidence of some types of hypersensitivity may be correlated with others. For example, it has been proposed that individuals with certain common allergies are more susceptible to autoimmune disorders.

### IV. Examples

The following examples are illustrative of the method and are in no way intended to be limiting.

### Example 1

A synthetic IFN-τ gene is generated using standard molecular methods. (Ausubel, et al., in CURRENT PROTOCOLS IN MOLECULAR BIOLOGY, John Wiley & Sons, Inc., Media, Pa. (1988)) by ligating oligonucleotides containing contiguous portions of a DNA sequence encoding the IFN-τ amino acid sequence. The DNA sequence used may be either SEQ ID NO:1, SEQ ID NO:2 or SEQ ID NO:3, variants thereof, or the sequence as shown in Imakawa, K. et al., Nature, 330:377-379, (1987). The resulting IFN-τ polynucleotide coding sequence may span position 16 through 531 to yield a coding sequence of 172 amino acids.

### Example 2

The full length synthetic gene StuI/SstI fragment (540 bp) was cloned into a modified pIN III omp-A expression vector and transformed into a competent SB221 strain of E. coli. To express the IFN-τ protein, cells carrying the expression vector were grown in L-broth containing ampicillin to an OD (550 nm) of 0.1-1, induced with IPTG (isopropyl-1-thio-β-D-galactoside) for 3 hours and harvested by centrifugation. Soluble recombinant IFN-τ was liberated from the cells by sonication or osmotic fractionation.

For expression in yeast, the IFN-τ gene was amplified using polymerase chain reaction (PCR; Mullis, K. B., U.S. Pat. No. 4,683,202, issued 28 Jul. 1987; Mullis, K. B., et al., U.S. Pat. No. 4,683,195, issued 28 Jul. 1987) with PCR primers containing StuI and SacI restriction sites at the 5′ and 3′ ends, respectively. The amplified fragments were digested with StuI and SacI and ligated into the SacII and SmaI sites of pBLUESCRIPT+(KS), generating pBSY-inteferon-tau. Plasmid pBSY-IFN-τ was digested with SacII and EcoRV and the fragment containing the synthetic IFN-τ gene was isolated. The yeast expression vector pBS24Ub (Ecker, D. J., et al., J. Biol. Chem. 264:7715-7719(1989)) was digested with SacII. Blunt ends were generated using T4 DNA polymerase. The vector DNA was extracted with phenol and ethanol precipitated (Sambrook, J., et al., in MOLECULAR CLONING: A LABORATORY MANUAL, Second Edition, Cold Spring Harbor Laboratory, Cold Spring Harbor, N.Y. (1989)). The recovered plasmid was digested with SacII, purified by agarose gel electrophoresis, and ligated to the SacII-EcoRV fragment isolated from pBSY-IFN-τ. The resulting recombinant plasmid was designated pBS24Ub-IFN-τ.

The recombinant plasmid pBS24Ub-inteferon-tau was transformed into E. coli. Recombinant clones containing the IFN-τ insert were isolated and identified by restriction enzyme analysis. IFN-τ coding sequenes were isolated from pBS24Ub-IFN-τ and cloned into a Pichia pastoris vector containing the alcohol oxidase (AOX1) promoter (Invitrogen, San Diego, Calif.). The vector was then used to transform Pichia pastoris GS115 His.sup.-host cells and protein was expressed following the manufacturer's instructions. The protein was secreted into the medium and purified by successive DEAE-cellulose and hydroxyapatite chromatorgraphy to electrophoretic homogeneity as determined by SDS-PAGE and silver staining.

### Example 3

Eight New Zealand White mice are obtained and experimental allergic encephalomyelitis is induced, as described in U.S. Pat. No. 6,060,450. Four of the mice are treated with interferon-τ applied intranasally daily for one month. During the treatment period, the severity of disease was graded on the following scale: 1, loss of tail tone; 2, hind limb weakness; 3, paraparesis; 4, paraplegia; 5, moribund/death. Animals treated with intranasally applied interferon-τ have a lower score than untreated animals.

### Example 4

Eight New Zealand White mice are obtained and experimental allergic encephalomyelitis is induced, as described in U.S. Pat. No. 6,060,450. Four of the mice are treated with interferon-τ applied mucosally via bronchial lavage to the lungs daily for one month. During the treatment period, the severity of disease was graded on the following scale: 1, loss of tail tone; 2, hind limb weakness; 3, paraparesis; 4, paraplegia; 5, moribund/death. Animals treated with intranasally applied interferon-τ have a lower score than untreated animals.

### Example 5

A study was undertaken to analyze gene expression in non-diseased (i.e., normal) mice after nasal administration of IFN-τ, to determine, e.g., whether IFN-τ, could modulate the immune environment in animal spleens.

Materials and Methods

Animals: 9-week old female SJL mice were obtained from Harlan (Indianapolis, Ind., USA). Mice were maintained on a 12 hour/12 hour diurnal light cycle. Standard rodent chow (Harlan Teklad, CAT# 8728C) and autoclaved commercial drinking water (Arrowhead) were supplied ad libido.

IFN-τ administration: Phosphate buffered saline (PBS) or IFN-τ (3.125 thousand units (KU), 12.5 KU or 50 KU) was administered in a volume of 4 μl delivered to each nostril using with a pipette tip in the morning of each day of administration. IFN-τ was diluted in PBS (pH7.4) immediately before the delivery. Four to 5 mice were included in each group.

Tissue Collection: Twenty four hours after IFN-τ administration, mice were euthanized by CO₂ asphyxiation. Spleens of the euthanized mice were dissected, snap-frozen in liquid nitrogen and stored at −80° C.

Gene Expression Analyses

Individual tissues were homogenized using a homogenizer in 3 ml Trizol reagent (Invitrogen, Carlsbad, Calif., USA; CAT# 19956-018). RNA was isolated according to the manufacturer's instructions. cDNA was synthesized using 20 ng RNA per 1 μl volume and the TaqMan Reverse Transcription Reagents (Applied Biosystems, Foster City, Calif., USA; CAT# N8080234). PCR reactions were setup using the gene expression assays for each gene (see Table 1) and the TaqMan Universal PCR Master Mix (Applied Biosystems, CAT# 432-4020). Each 25 μl PCR reaction included the cDNA reverse-transcribed from 30 ng RNA.

Real Time PCR was performed on a 7500 real time PCR system (Applied Biosystems, Inc). Relative quantification (RQ) analysis was performed using the system software and the hprt (hypoxanthine-guanine phosphoribosyl transferase) gene as an endogenous control. The level of hprt expression was defined as 1 and the levels of expression of other genes was described relative to this amount. Paired student t test (assuming equal variance) was used to assess the significance of differences in gene expression in control (PBS-treated) animals and IFN-τ-treated animals.

Results and Discussion

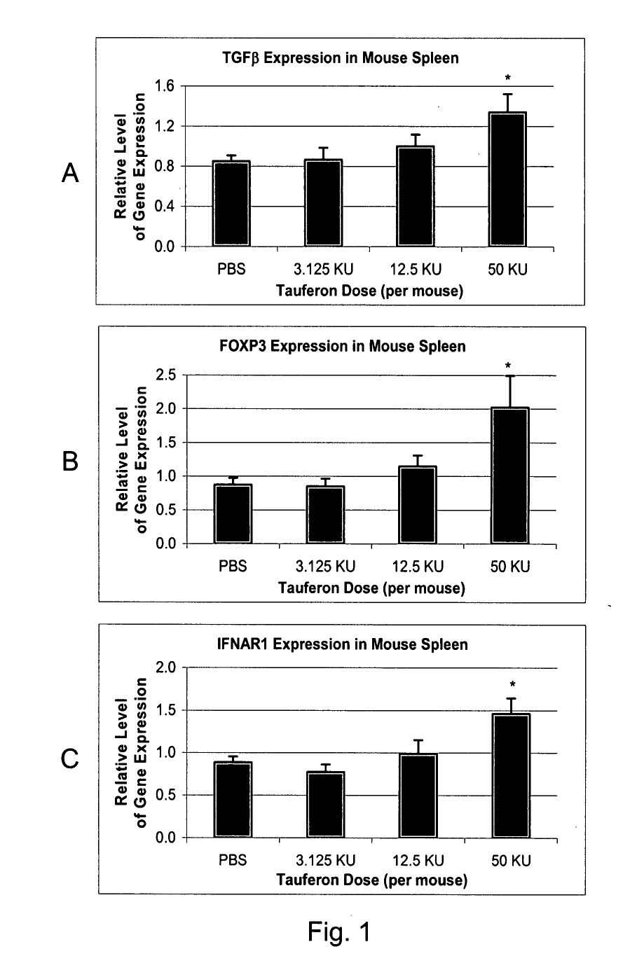

Eleven genes (i.e., CCR3, FOXP3, IFNAR1, IFNAR2, IFNγ, IL-10, IL-4, INDOMX1, OAS1A, TGFβ, and Hprt) were examined for their expression in murine spleens at 24 hours after nasal delivery of IFN-τ, compared with control animals (FIGS. 1A-1C and Table 2). Of these genes, TGFβ, FOXP3, and IFNAR1 were most affected by IFN-τ administration.

Analyses of the real time PCR results showed that TGFβ was upregulated by IFN-τ in a dose-dependent manner when mice were treated with 3.125, 12.5, or 50 KU IFN-τ (FIG. 1A). The average mRNA levels in the 50 KU IFN-τ-dosed mice was significantly higher (58%) than in the PBS control mice. TGFβ plays a crucial role in immune suppression (Li, M. O. et al. (2006) Transforming growth factor-beta regulation of immune responses. Annu Rev Immunol. 24:99-146; Bonecchi, R. et al. (1998) Differential expression of chemokine receptors and chemotactic responsiveness of type 1 T helper cells (Th1s) and Th2s. J. Exp. Med. 187:129-134). It has been reported that TGFβ mediates the anti-inflammatory of IFN-τ, at inflammation sites in experimental autoimmune encephalomyelitis, a mouse model for multiple sclerosis and other diseases. TGFβ expression was found to be up-regulated by IFN-τ, in a mouse model for inflammatory bowel disease.

Analyses of the real time PCR results showed that FOXP3 was upregulated by IFN-τ in a dose-dependent manner when mice were treated with 3.125, 12.5, or 50 KU IFN-τ (FIG. 1B). The average level of FOXP3 expression was increased 130% in 50 KU IFN-τ-treated mice, significantly different from that of the PBS mice. FOXP3 is a marker for CD4+/CD25+ T-cells (Bluestone and Tang (2005) How do CD4+ CD25+ regulatory T cells control autoimmunity? Curr Opin Immunol. 17:638-42).

Analyses of the real time PCR results showed that IFNAR1 was upregulated by IFN-τ in a dose-dependent manner when mice were treated with 3.125, 12.5, or 50 KU IFN-τ (FIG. 1C). The average level of IFNAR1 expression was increased 65% in 50 KU IFN-τ-treated mice, significantly different from that of the PBS mice. IFNAR1 is a gene encoding one of the two subunits of the Type 1 interferon receptor, suggesting that the regulation of the TGFβ and FOXP3 genes by IFN-τ may be mediated by the Type 1 interferon receptor (Grohmann, U. et al. (2003) Tolerance, DCs and tryptophan: much ado about IDO. Trends Immunol. 24:242-8).

Taken together, these gene expression results suggested that nasal administration is an effective route of delivery for of IFN-τ for purpose of systemic suppression of T-cell immunity. The data further suggest that the Type 1 interferon receptor may mediate the immunomodulatory activities observed with of IFN-τ.

TABLE 1
Genes selected for expression analysis.
The primers (i.e., probe set) in each assay produced an amplicon from
multi-exons.

| | Gene Expression Assay# |
|---|---|
| Gene | (Applied Biosystems) |
| CCR3 | Mm01216172_m1 |
| FOXP3 | Mm00475156_m1 |
| IFNAR1 | Mm00439544_m1 |
| IFNAR2 | Mm00494916_m1 |
| IFNγ | Mm00801778_m1 |
| IL-10 | Mm00439616_m1 |
| IL-4 | Mm00445259_m1 |
| INDO | Mm00492586_m1 |
| MX1 | Mm00487796_m1 |
| OAS1A | Mm00836412_m1 |
| TGFβ | Mm00441724_m1 |
| Hprt | Mm00446968_m1 |

TABLE 2
Gene Expression in Spleens of Mice at 24 Hours after Nasal
Administration of Tauferon. Hprt gene was used as an internal
control for gene expression.

| Sample ID | Tauferon | TGFβ | FOXP3 | IFNAR1 |
|---|---|---|---|---|
| Spleen IIIA1 | PBS | 1 | 1 | 1 |
| Spleen IIIA2 | PBS | 0.728 | 0.624 | 0.69 |
| Spleen IIIA3 | PBS | 0.958 | 0.79 | 0.758 |
| Spleen IIIA4 | PBS | 0.733 | 1.193 | 1.017 |
| Spleen IIIA5 | PBS | 0.826 | 0.784 | 0.962 |
| Average | | 0.849 | 0.878 | 0.885 |
| STD | | 0.126 | 0.221 | 0.151 |
| SE | | 0.056 | 0.099 | 0.067 |
| Spleen IIIB1 | 3.125 KU | 0.802 | 0.581 | 0.619 |
| Spleen IIIB2 | 3.125 KU | 0.851 | 0.866 | 0.627 |
| Spleen IIIB3 | 3.125 KU | 1.187 | 1.131 | 0.882 |
| Spleen IIIB4 | 3.125 KU | 0.617 | 0.833 | 0.961 |
| Average | | 0.864 | 0.853 | 0.772 |
| STD | | 0.238 | 0.225 | 0.175 |
| SE | | 0.119 | 0.112 | 0.088 |
| Change | | 2% | −3% | −13% |
| P Value | | 0.90 | 0.87 | 0.33 |
| Spleen | 12.5 KU | 0.909 | 0.805 | 0.633 |
| IIIC1 | | | | |
| Spleen | 12.5 KU | 1.034 | 1.318 | 1.043 |
| IIIC2 | | | | |
| Spleen | 12.5 KU | 0.762 | 1.496 | 1.401 |
| IIIC3 | | | | |
| Spleen | 12.5 KU | 1.302 | 0.966 | 0.875 |
| IIIC4 | | | | |
| Average | | 1.002 | 1.146 | 0.988 |
| STD | | 0.229 | 0.317 | 0.323 |
| SE | | 0.114 | 0.158 | 0.161 |
| Change | | 18% | 31% | 12% |
| P Value | | 0.24 | 0.18 | 0.54 |
| Spleen | 50 KU | 1.346 | 1.46 | 1.164 |
| IIID1 | | | | |
| Spleen | 50 KU | 1.245 | 1.094 | 1.226 |
| IIID2 | | | | |
| Spleen | 50 KU | 0.948 | 2.321 | 1.507 |
| IIID3 | | | | |
| Spleen | 50 KU | 1.825 | 3.208 | 1.953 |
| IIID4 | | | | |
| Average | | 1.341 | 2.021 | 1.463 |
| STD | | 0.364 | 0.944 | 0.359 |
| SE | | 0.182 | 0.472 | 0.180 |
| Change | | 58% | 130% | 65% |
| P Value | | 0.02 | 0.03 | 0.01 |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A treatment method, comprising
administering an effective amount of an interferon therapeutic agent to a respiratory tract region to treat a condition responsive to interferon therapy.

2. The method of claim 1, wherein said administering comprises administering a Type I interferon or a Type II interferon.

3. The method of claim 1, wherein said administering comprises administering an interferon selected from the group of interferon-alpha, interferon-beta, interferon-omega, interferon-tau and variant interferon.

4. The method of claim 1, wherein said administering comprises administering said therapeutic agent in the form of a solution.

5. The method of claim 1, wherein said administering comprises administering said therapeutic agent in the form of a suspension.

6. The method of claim 1, wherein said administering comprises administering said therapeutic agent in the form of an ointment.

7. The method of claim 1, wherein said administering comprises administering said therapeutic agent in the form of an aerosol.

8. The method of claim 1, wherein said administering comprises administering said therapeutic agent in the form of dry powder.

9. The method of claim 8, wherein said administering is accomplished using a dry powder inhalation device.

10. The method of claim 1, wherein said administering comprises administering said therapeutic agent in a sustained release dosage form.

11. The method of claim 1, wherein said administering comprises administering an interferon-tau having a sequence at least 80% identical to SEQ ID NO:2.

12. The method of claim 1, wherein said administering comprises administering an interferon-tau having a sequence identified herein as SEQ ID NO:1 or SEQ ID NO:2.

13. The method of claim 1, wherein said administering comprises administering a variant interferon having a sequence identified herein as SEQ ID NO:3.

14. The method of claim 1, wherein said administering comprises administering a composition comprising histidine.

15. The method of claim 1, wherein said condition comprises a systemic disease.

16. The method of claim 1, further comprising administering a second therapeutic agent to the patient.

17. The method of claim 16, wherein said second therapeutic agent is administered by a means selected from the group of oral, nasal, mucosal, parenteral and topical means.

18. The method of claim 1, wherein said respiratory tract region is the upper respiratory tract.

19. The method of claim 1, wherein said respiratory tract region is the lower respiratory tract.

20. The method of claim 1, wherein said respiratory tract region is the nasal cavity.

21. The method of claim 1, wherein said respiratory tract region is the bronchioles.

22. The method of claim 1, wherein said respiratory tract region is the alveoli.

23. The method of claim 1, wherein the condition responsive to interferon therapy is a disease selected from multiple sclerosis, type I (insulin dependent) diabetes mellitus, lupus erythematosus, amyotrophic lateral sclerosis, Crohn's disease, rheumatoid arthritis, stomatitis, asthma, uveitis, allergies, and psoriasis.

24. The method of claim 23, wherein the interferon is interferon-tau.

25. The method of claim 24, wherein administration of interferon-tau causes systemic suppression of T-cell immunity.

26. The method of claim 25, wherein administration of interferon-tau causes an increase in the levels of a gene selected from TGFβ, FOXP3, and IFNAR1.


SEQUENCE LISTING (3 sequences)

SEQ ID NO: 1 - protein, 172 aa, Ovis aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 2 - protein, 172 aa, Artificial Sequence
  Modified Amino acid sequence of mature ovine IFN-tau
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 3 - protein, 166 aa, Artificial Sequence
  Amino acid sequence of the IFN-alpha analog IFN-alpha-N7
CDLPETHSLDNRRTLMLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE